(19) United States
(12) Patent Application Publication
(10) Pub. No.: US 2022/0160310 A1
(43) Pub. Date: May 26, 2022

(54) SYMPTOM LOGGER

(71) Applicant: Medtronic, Inc., Minneapolis, MN (US)

(72) Inventors: Pranam Shetty, Plymouth, MN (US); Niranjan Chakravarthy, Singapore (SG); Maneesh Shrivastav, Blaine, MN (US); Rodolphe Katra, Blaine, MN (US); Thomas Piaget, St. Paul, MN (US); Arthur K. Lai, Minnetonka, MN (US)

(21) Appl. No.: 17/103,432

(22) Filed: Nov. 24, 2020

(51) Int. Cl. A61B 5/00; G16H 40/67; G16H 10/60

(52) U.S. Cl. CPC A61B 5/7275; G16H 10/60; G16H 40/67; A61B 5/74

(57) ABSTRACT

This disclosure is directed to techniques for recording and recognizing physiological parameter patterns associated with symptoms. A medical device system includes a medical device including one or more sensors configured to generate a signal that indicates a parameter of a patient. Additionally, the medical device system includes processing circuitry configured to receive data indicative of a user indication of an experienced symptom; determine a plurality of parameter values of the parameter based on a portion of the signal corresponding to a period of time including a time before the user indication and a period of time after the user indication. Additionally, the processing circuitry is configured to identify, based on a reference set of parameter values of the plurality of parameter values, the experienced symptom. Additionally, the processing circuitry is configured to save, to a database in memory, a set of data including the experienced symptom and patient parameters.

### TECHNICAL FIELD

The disclosure relates generally to medical device systems and, more particularly, medical device systems configured to monitor and record patient parameters.

### BACKGROUND

Some types of medical devices may be used to monitor one or more physiological parameters of a patient. Such medical devices may include, or may be part of a system that includes, sensors that detect signals associated with such physiological parameters. Values determined based on such signals may be used to assist in detecting changes in patient conditions, in evaluating the efficacy of a therapy, or in generally evaluating patient health.

### SUMMARY

In general, the disclosure is directed to devices, systems, and techniques for recording and recognizing physiological parameter patterns associated with patient symptoms. For example, a medical device, e.g., an implantable medical device (IMD), may collect one or more signals which include one or more values of a parameter (e.g., a physiological parameter) of a patient for a period of time before the patient indicates that the patient is experiencing symptoms of a condition. A medical device may also collect one or more values of the parameter(s) for a period of time after the symptom indication. Processing circuitry of a system that includes the medical device may record the time course of parameter values, or information representative of the time course of parameter values, in a database as corresponding to the symptom reported by the patient.

Based on the signal, the processing circuitry and algorithms may identify, from among a number of diseases present in the patient, a disease that corresponds to the experienced symptom and the collected parameter values. The processing circuitry may further compare future parameter value patterns to the database, and when sufficiently similar patterns are detected, automatically take a number of actions, such as collect and record the new parameter values in the database corresponding to the symptom, as well as prompt the patient to enter additional information regarding the symptom and/or their condition. The processing circuitry may foreworn the patient, based on sufficiently similar patterns, when the patient may be expected to experience a symptom.

The techniques of this disclosure may provide one or more advantages. For example, it may be beneficial for a physician to have specific parameter information for the patient while the patient experiences symptoms. A data log of specific parameter values associated with symptoms, and contemporaneous information from the patient regarding the symptoms, can be more accurate, comprehensive, and specific than patient-reported parameter values or other patient-reported symptom information collected using conventional techniques. Patients may fail to keep a record of their symptoms, fail to record all their symptoms, or fail to accurately record what symptoms were experienced. By the time patients speak with their physicians, they may have forgotten certain experienced symptoms. Even if patients are wearing a medical device, they may fail to prompt the medical device to record parameter values when they experience a symptom. Ambiguous patient-reported symptoms can be non-informative diagnostically and take up caregiver resources. The techniques of this disclosure may provide further advantage by using a device to automatically warn a patient of certain impending symptoms. The device may prevent harm to the patient by allowing the patient to adjust his or her person or surroundings to prepare for the symptom.

In some examples, a medical device system includes a medical device including one or more sensors configured to sense one or more signals that indicate one or more parameters of a patient; and processing circuitry configured to: receive a patient indication of an occurrence of a symptom; determine a time period based on the patient indication, determine a plurality of parameter values of the one or more parameters of the patient during the time period; and save, to a database in memory, a set of data including the determined patient parameter values.

In some examples, a medical device system includes a medical device including one or more sensors configured to sense one or more signals that indicate one or more parameters of a patient; and processing circuitry configured to: determine a plurality of parameter values of the one or more parameters of the patient during a time period; compare the determined parameter values to reference data sets associated with one or more symptoms or impending symptoms from the database in memory determine that a sufficient match exists between the determined parameter values and one of the reference data sets; responsive to determining that the sufficient match exists, notify the patient of the symptom associated with the one of the reference data sets; and receive a patient confirmation or denial of the notified symptom. The reference set of parameter values may be, for example, a population-based distribution corresponding to the experienced symptom, or patient-specific data for the experienced symptom.

In some examples, a method includes sensing, by a medical device including one or more sensors, one or more signals that indicate one or more parameters of a patient; receiving, by processing circuitry, a patient indication of an occurrence of a symptom; determining, by the processing circuitry, a time period based on the patient indication; determining, by the processing circuitry, a plurality of parameter values of the one or more parameters of the patient during the time period; and saving, by the processing circuitry to a database in memory, a set of data including the determined patient parameters.

In some examples, a method includes sensing, by a medical device including one or more sensors, one or more signals that indicate one or more parameters of a patient; determining, by processing circuitry, a plurality of parameter values of the one or more parameters of the patient during a time period; comparing, by the processing circuitry, the determined parameter values to reference data sets associated with one or more symptoms or impending symptoms from the database in memory; determining, by the processing circuitry, that a sufficient match exists between the determined parameter values and one of the reference data sets; responsive to determining that the sufficient match exists, notifying, by the processing circuitry, the patient of the symptom associated with the one of the reference data sets; and receiving, by the processing circuitry, a patient confirmation or denial of the notified symptom. The reference set of parameter values may be, for example, a population-based distribution corresponding to the experienced symptom, or patient-specific data for the experienced symptom.

In some examples, a non-transitory computer-readable medium includes instructions for causing one or more processors to: sense one or more signals that indicate one or more parameters of a patient; receive a patient indication of an occurrence of a symptom; determine a time period based on the patient indication, determine a plurality of parameter values of the one or more parameters of the patient during the time period; and save, to a database in memory, a set of data including the determined patient parameter values.

In some examples, a non-transitory computer-readable medium includes instructions for causing one or more processors to sense one or more signals that indicate one or more parameters of a patient; determine a plurality of parameter values of the one or more parameters of the patient during a time period; compare the determined parameter values to reference data sets associated with one or more symptoms or impending symptoms from the database in memory determine that a sufficient match exists between the determined parameter values and one of the reference data sets; responsive to determining that the sufficient match exists, notify the patient of the symptom associated with the one of the reference data sets; and receive a patient confirmation or denial of the notified symptom. The reference set of parameter values may be, for example, a population-based distribution corresponding to the experienced symptom, or patient-specific data for the experienced symptom.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a conceptual drawing illustrating an example configuration of the implantable medical device (IMD) of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIGS. 4A and 4B illustrate two additional example IMDs that may be substantially similar to the IMD of FIGS. 1-3, but which may include one or more additional features, in accordance with one or more techniques described herein.

FIG. 5 is a block diagram illustrating an example configuration of components of the external device of FIG. 1, in accordance with one or more techniques of this disclosure.

FIG. 6 is a block diagram illustrating an example system that includes an access point, a network, external computing devices, such as a server, and one or more other computing devices, which may be coupled to the IMD, the external device, and the processing circuitry of FIG. 1 via a network, in accordance with one or more techniques described herein.

FIG. 7 is a flow diagram illustrating an example operation for enhancing the information yield and specificity of symptom information based on a user selection of a reference time, in accordance with one or more techniques of this disclosure.

FIG. 8 is a flow diagram illustrating an example operation for obtaining and identifying disease-specific symptom information based on a user selection of a reference time, in accordance with one or more techniques of this disclosure.

FIG. 9 is a flow diagram illustrating an example operation for identifying, predicting, and notifying a patient of impending symptoms, in accordance with one or more techniques of this disclosure.

Like reference characters denote like elements throughout the description and figures.

### DETAILED DESCRIPTION

This disclosure describes techniques for logging and recalling one or more parameters of a patient and matching those parameters to symptoms and diseases. Patients often do not record symptoms of diseases when they experience them. When they do, they often forget the symptom or its precursors, mistake one symptom for another, or miss a symptom entirely when experiencing multiple at a time. For example, in January 2017 alone, 761 patients using a SEEQ Mobile Cardiac Telemetry (MCT) System indicated a symptomatic episode 4933 times. Yet symptom information was not available in around 59% of those instances nor for around 37% of the patients. In some examples, it may be beneficial to record a data set including patient parameters corresponding to symptoms so that a treating physician has accurate and specific information off which to base a treatment plan or diagnosis. Additionally, it may be beneficial to monitor the patient parameters and compare them to known symptom events in order to predict when a symptom event will occur and prepare the patient for that event.

It may be especially beneficial to record data sets including patient parameters corresponding to symptoms in patients with comorbidities to help identify which condition is causing the symptoms. Identification is beneficial, as comorbidities may have very different treatment and therapy programs, for example arrhythmias may be treated with a pacemaker and chronic obstructive pulmonary disease (COPD) may be treated with oxygen therapy. In order to identify which comorbidity is causing the symptoms, it may be beneficial to record data sets including patient parameters that are not easily perceived by a patient. Comorbidities may manifest physically in subtly different ways but be experienced by patients very similarly. For example, arrhythmias are a common comorbidity with COPD, both of which may be experienced by a patient as shortness of breath. However, arrhythmia and COPD may be distinguished by other patient parameter measurements like heart rate.

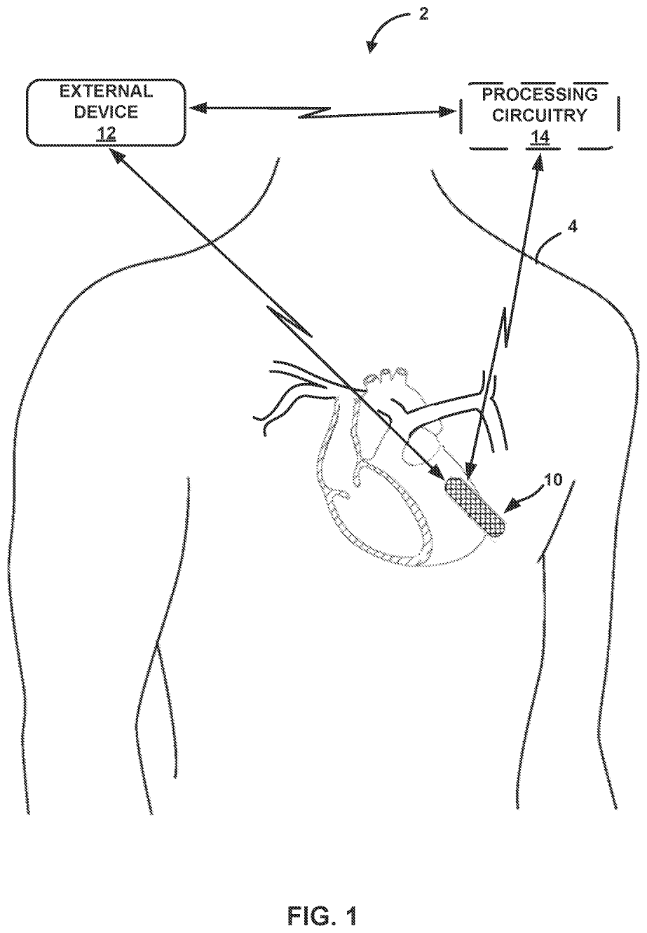

FIG. 1 is a conceptual diagram illustrating an environment of an example medical device system 2 in conjunction with a patient 4, in accordance with one or more techniques of this disclosure. The example techniques may be used with an IMD 10, which may be in wireless communication with at least one of external device 12 and other devices not pictured in FIG. 1. Processing circuitry 14 is conceptually illustrated in FIG. 1 as separate from IMD 10 and external device 12 but may be processing circuitry of IMD 10 and/or processing circuitry of external device 12. In general, the techniques of this disclosure may be performed by processing circuitry 14 of one or more devices of a system, such as one or more devices that include sensors that provide signals, or processing circuitry of one or more devices that do not include sensors, but nevertheless analyze signals using the techniques described herein. For example, another external device (not pictured in FIG. 1) may include at least a portion of processing circuitry 14, the other external device configured for remote communication with IMD 10 and/or external device 12 via a network.

In some examples, IMD 10 is implanted outside of a thoracic cavity of patient 4 (e.g., subcutaneously in the pectoral location illustrated in FIG. 1). IMD 10 may be positioned near the sternum near or just below the level of patient 4's heart, e.g., at least partially within the cardiac silhouette. In some examples, IMD 10 takes the form of a LINQ™ Insertable Cardiac Monitor (ICM), available from Medtronic plc, of Dublin, Ireland.

Although in one example IMD 10 takes the form of an ICM, in other examples, IMD 10 takes the form of any combination of implantable cardiac devices (ICDs) with intravascular or extravascular leads, pacemakers, cardiac resynchronization therapy devices (CRT-Ds), neuromodulation devices, left ventricular assist devices (LVADs), implantable sensors, cardiac resynchronization therapy pacemakers (CRT-Ps), implantable pulse generators (IPGs), orthopedic devices, or drug pumps, as examples. Moreover, techniques of this disclosure may be used to measure one or more patient parameters based on signals collected by one or more of the aforementioned devices. Additionally, or alternatively, techniques of this disclosure may be used to measure one or more patient parameters based on signals collected by one or more external devices such as patch devices, wearable devices (e.g., smart watches), wearable sensors, or any combination thereof.

Clinicians sometimes diagnose patients with medical conditions based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patent is in a clinic for a medical appointment. However, in some examples, physiological markers (e.g., irregular heartbeats and long-term respiration trends) of a patient condition occur when the patient is outside the clinic. As such, in these examples, a clinician may be unable to observe the physiological markers needed to diagnose a patient with a medical condition. Additionally, it may be beneficial to monitor one or more patient parameters for an extended period of time (e.g., days, weeks, or months) so that the one or more parameters may be analyzed to identify a patient's unique physiological markers that accompany a symptom or medical condition. In the example illustrated in FIG. 1, IMD 10 is implanted within patient 4 to continuously record one or more physiological signals of patient 4 over an extended period of time.

IMD 10 may include any one or more electrodes, optical sensors, motion sensors (e.g., accelerometers), temperature sensors, chemical sensors, pressure sensors, or any combination thereof and any additional sensors that may be a part of IMD 10. Such sensors may sense one or more signals that indicate one or more physiological parameters of a patient. The one or more physiological parameters of the patient may be indicative of a patient condition, including a symptom or disease. Various features may be extracted from sensor signals, for example: the amount of deviation from a baseline; the timing of the deviation; absolute values corresponding to physiological parameters of a patient (e.g., a heart rate of 80 bpm).

In some examples, IMD 10 includes one or more sensor(s) which are configured to detect physiological signals of patient 4. For example, IMD 10 includes a set of electrodes (not illustrated in FIG. 1). The set of electrodes are configured to detect one or more signals associated with cardiac functions and/or lung functions of patient 4. In some examples, IMD 10 may sense an electrogram (EGM) via the set of electrodes. The EGM may represent one or more physiological electrical signals corresponding to the heart of patient 4. For example, the EGM may indicate ventricular depolarizations (R-waves), atrial depolarizations (P-waves, ventricular Repolarizations (T-waves), among other events. Information relating to the aforementioned events, such as time separating one or more of the events, may be applied for a number of purposes, such as to determine whether an arrhythmia is occurring, predict whether an arrhythmia is likely to occur, and/or determine a number of premature ventricular contractions (PVCs). Cardiac signal analysis circuitry, which may be implemented as part of processing circuitry 14, may perform signal processing techniques to extract information indicating the one or more parameters of the cardiac signal.

In some examples, the IMD 10 may be configured to detect a tissue impedance signal via the set of electrodes. The tissue impedance signal may represent a resistance value between one or more of the set of electrodes and subcutaneous tissue of patient 4. The tissue impedance may be applied for a number of purposes, such as to determine whether an arrhythmia is occurring and/or predict whether an arrhythmia is likely to occur, or to determine a level of perfusion, edema, respiration rate, effort and pattern, and/or heart failure.

IMD 10 may include an optical sensor. The optical sensor may, in some cases, include two or more light emitters and one or more light detectors. The optical sensor may perform one or more measurements in order to determine an oxygenation of the tissue of Patient 4. For example, the optical sensor may perform one or more tissue oxygen saturation (StO₂) measurements. StO₂ may, in some examples, represent a weighted average between Arterial blood oxygen saturation (SaO₂) and venous oxygen saturation (SvO₂). In some examples, the optical sensor may perform one or more pulse oximetry (SpO₂) measurements. SpO₂ may, in some cases, represent an approximation of SaO₂. Oxygen saturation (e.g., StO₂, SaO₂, SvO₂, and SpO₂) trends may be indicative of one or more patient conditions, such as heart failure, sleep apnea, or COPD, as examples. For example, a steady decline of StO₂ values over a period time may indicate a worsening risk of a heart failure exacerbation in a patient. As such, the IMD may perform several StO₂ measurements over a period of time (e.g., hours, days, weeks, or months) and the processing circuitry may identify a trend of StO₂ values using data from the StO₂ measurements. Based on the identified trend, the processing circuitry may, in some cases, identify a medical condition present in the patient or monitor a condition that is already known to be present in the patient.

During the respective StO₂ measurement, the light emitters of the optical sensor may output light to an area of tissue proximate to the IMD, the light including a first set of frequency components. The one or more light detectors may sense light including a second set of frequency components. The processing circuitry is configured to compare the first set of frequency components and the second set of frequency components to identify an StO₂ value corresponding to the respective StO₂ measurement, where the StO₂ value represents a ratio of oxygen-saturated hemoglobin located in the area of tissue to a total amount of hemoglobin located in the area of tissue.

In some examples, IMD 10 includes one or more accelerometers. An accelerometer of IMD 10 may collect an accelerometer signal which reflects a measurement of a motion and/or posture of patient 4. In some cases, the accelerometer may collect a three-axis accelerometer signal indicative of patient 4's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 4 along a vertical axis, the lateral axis accelerometer signal vector may represent an acceleration of patient 4 along a lateral axis, and the frontal axis accelerometer signal vector may represent an acceleration of patient 4 along a frontal axis. In some cases, the vertical axis substantially extends along a torso of patient 4 from a neck of patient 4 to a waist of patient 4, the lateral axis extends across a chest of patient 4 perpendicular to the vertical axis, and the frontal axis extends outward from and through the chest of patient 4, the frontal axis being perpendicular to the vertical axis and the lateral axis.

An IMD may include one or more electrodes configured to measure an electrogram (EGM) of the patient. The EGM may, in some cases, indicate a ventricular depolarization (e.g., an R-wave) of the patient's heart and a heart rate of the patient. Additionally, the IMD may determine tissue perfusion based on impedance sensed via the electrodes, and/or oxygen saturation using an optical sensor. Processing circuitry may determine a pulse transit time (PTT) associated with the patient based on the EGM, the impedance, the measured oxygen saturation, or any combination thereof. PTT is correlated with blood pressure. As such, processing circuitry may be configured to use a PTT measurement performed by the IMD as a representation of the blood pressure of the patient. In this way, the processing circuitry may be configured to track the blood pressure and the heart rate of the patient over a period of time.

External device 12 may be a computing device configured for use in settings such as a home, clinic, or hospital, and may further be configured to communicate with IMD 10 via wireless telemetry. For example, external device 12 may be coupled to a remote patient monitoring system, such as Carelink®, available from Medtronic plc, of Dublin, Ireland. External device 12 may, in some examples, include a programmer, an external monitor, or a consumer device such as a smart phone or tablet.

In other examples, external device 12 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device.

When external device 12 is configured for use by the clinician, external device 12 may be used to transmit instructions to IMD 10. Example instructions may include requests to set electrode combinations for sensing and any other information that may be useful for programming into IMD 10. The clinician may also configure and store operational parameters for IMD 10 within IMD 10 with the aid of external device 12. In some examples, external device 12 assists the clinician in the configuration of IMD 10 by providing a system for identifying potentially beneficial operational parameter values.

Whether external device 12 is configured for clinician or patient use, external device 12 is configured to communicate with IMD 10 and, optionally, another computing device (not illustrated by FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies). In some examples, external device 12 is configured to communicate with a computer network, such as the Medtronic CareLink® Network developed by Medtronic, plc, of Dublin, Ireland. For example, external device 12 may send data, such as data received from IMD 10, to another external device such as a smartphone, a tablet, or a desktop computer, and the other external device may in turn send the data to the computer network. In other examples, external device 12 may directly communicate with the computer network without an intermediary device.

Processing circuitry 14, in some examples, may include one or more processors that are configured to implement functionality and/or process instructions for execution within IMD 10, external device 12, one or more other devices, or any combination thereof. For example, processing circuitry 14 may be capable of processing instructions stored in a memory. Processing circuitry 14 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 14 may include any suitable structure, whether in hardware, software, firmware, or any combination thereof, to perform the functions ascribed herein to processing circuitry 14.

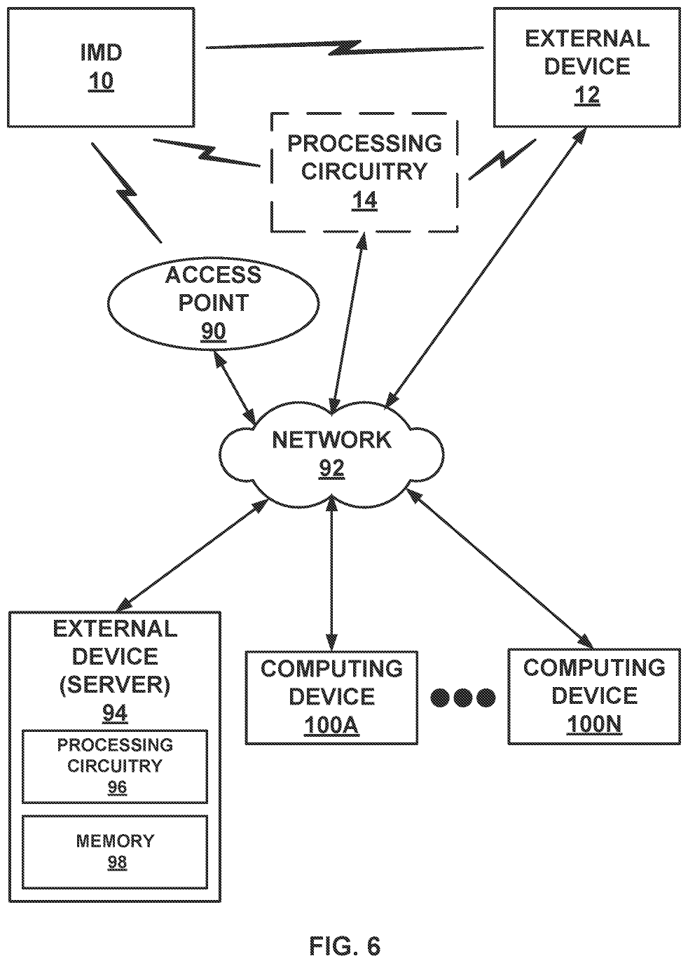

Processing circuitry 14 may represent processing circuitry located within any combination of IMD 10 and external device 12. In some examples, processing circuitry 14 may be entirely located within a housing of IMD 10. In other examples, processing circuitry 14 may be entirely located within a housing of external device 12. In other examples, processing circuitry 14 may be located within any combination of IMD 10, external device 12, and another device or group of devices that are not illustrated in FIG. 1. As such, techniques and capabilities attributed herein to processing circuitry 14 may be attributed to any combination of IMD 10, external device 12, and other devices that are not illustrated in FIG. 1, e.g., one or more servers or computing devices as illustrated with respect to FIG. 6.

A memory (not illustrated in FIG. 1) may be configured to store information within medical device system 2 during operation. The memory may include a computer-readable storage medium or computer-readable storage device. In some examples, the memory includes one or both of a short-term memory or a long-term memory. The memory may include, for example, random-access memories (RAM), dynamic random-access memories (DRAM), static random-access memories (SRAM), magnetic discs, optical discs, flash memories, or forms of electrically programmable memories (EPROM) or electrically erasable and programmable memories (EEPROM). In some examples, the memory is used to store program instructions for execution by processing circuitry 14.

The memory may represent a memory located within any one or both of IMD 10 and external device 12. In some examples, the memory may be entirely located within a housing of IMD 10. In other examples, the memory may be entirely located within a housing of external device 12. In other examples, the memory may be located within any combination of IMD 10, external device 12, and another device or group of devices that are not illustrated in FIG. 1. As such, techniques and capabilities attributed herein to the memory may be attributed to any combination of IMD 10, external device 12, and other devices that are not illustrated in FIG. 1.

In some examples, one or more sensors (e.g., electrodes, motion sensors, optical sensors, temperature sensors, or any combination thereof) of IMD 10 may sense one or more signals that indicate a parameter or set of parameters of a patient. In some examples, the signal that indicates the parameter includes a plurality of parameter values, where each parameter value of the plurality of parameter values represents a measurement, e.g., periodic measurement, of the parameter at a respective interval of time. The plurality of parameter values may represent a sequence of parameter values, where each parameter value of the sequence of parameter values are collected by IMD 10 at a start of each time interval of a sequence of time intervals. For example, IMD 10 may perform a parameter measurement in order to determine a parameter value of the sequence of parameter values according to a recurring time interval (e.g., every day, every night, every other day, every twelve hours, every hour, or any other recurring time interval). In another example, IMD 10 may perform a parameter measurement in response to a patient notification that measurement should begin. In another example, IMD 10 may constantly perform parameter measurements. In this way, IMD 10 may be configured to track a respective patient parameter more effectively as a patient need not be in a clinic for a parameter to be tracked, since IMD 10 is implanted within patient 4 and is configured to perform parameter measurements according to recurring or other time intervals without missing a time interval.

IMD 10 may measure a set of parameters including an impedance (e.g., subcutaneous impedance, an intrathoracic impedance or an intracardiac impedance) of patient 4, a respiratory rate, effort and pattern of patient 4 during night hours, a respiratory rate, effort and pattern of patient 4 during day hours, a heart rate of patient 4 during night hours, a heart rate of patient 4 during day hours, an atrial fibrillation (AF) burden of patient 4, a ventricular rate of patient 4 while patient 4 is experiencing AF, a PVC count of patient 4, a body temperature of patient 4, oxygen saturation of tissues inside patient 4, a body position of patient 4, an activity level of patient 4, any other parameter any combination thereof.

Processing circuitry 14 may be configured to identify one or more patient parameters based on physiological signals measured by IMD 10 or other devices. In some examples, processing circuitry 14 may be configured to determine a heart rate of patient 4 based on the EGM signal measured via one or more electrodes of IMD 10. In some examples, processing circuitry 14 may determine a blood pressure of patient 4 or one or more values corresponding to the blood pressure of patient 4 based on an EGM, an impedance signal, a tissue perfusion signal (e.g., collected by an optical sensor), or any combination thereof. Additionally, or alternatively, processing circuitry 14 may determine a speed of one or more body position movements detected in the accelerometer signals, identify a stability of a gait identified in the accelerometer signal, detect falls or near falls in the accelerometer signals, determine one or more tissue perfusion values identified in the optical signal sensed by IMD 10 via the optical sensor, determine one or more other patient parameters based on signals collected by IMD 10 or other devices, or any combination thereof.

In some examples, to determine the heart rate of patient 4, processing circuitry 14 may determine the heart rate based on two or more R-waves detected in the EGM collected by IMD 10. For example, the EGM may include one or more R-waves each representing a ventricular depolarization of the heart of patient 4. The rate of R-waves in the EGM may represent the heart rate of patient 4. As such, processing circuitry 14 may determine the heart rate of patient 4 over a period of time by determining the rate of R-waves in the EGM over the period of time. In some examples, the processing circuitry 14 may determine an amount of time between a first R-wave and a second R-wave consecutive to the first-wave. Based on the amount of time between the first R-wave and the second R-wave, the processing circuitry 14 may determine a heart rate of the patient 4 at the time of the second R-wave. Processing circuitry 14 may calculate the respective heart rate corresponding to each pair of consecutive R-waves in the EGM. As such, processing circuitry 14 may monitor the heart rate of patient 4 over time.

Processing circuitry 14 may receive a portion of the signal that includes the plurality of parameter values. In this way, processing circuitry 14 may receive at least a portion of the sequence of parameter values such that processing circuitry 14 can analyze the signal in order to determine whether a similar set of parameter values has been recorded in a symptom database 66 in memory 56. Processing circuitry 14 may determine that a similar set of parameter values has been recorded in a symptom database 66 in memory 56 by identifying a sufficient match between the current signal and the recorded parameter values. In some examples, processing circuitry 14 may receive data indicative of a user indication of a symptom. Processing circuitry 14 may receive the data from external device 12 or another device, where the user selection is a patient selection of a time in which a symptom is being experienced. As described herein, the “time” in which a symptom is being experienced may refer to a point of time (e.g., an hour, a second, or a fraction of a second) in which the patient notifies the device of a symptom, a time just before the patient notifies the device of a symptom, and/or a time just after the patient notifies the device of a symptom. In some examples, IMD 10 may continuously collect parameter values at a predetermined frequency. IMD 10, a server, or another storage device may include a buffer or other memory structure which temporarily or permanently stores parameter values.

Processing circuitry 14 may maintain a symptom database which stores a plurality of sets of data in logs corresponding to a respective symptom. Processing circuitry 14 may also maintain a disease database which stores a plurality of sets of data each corresponding to a respective disease diagnosis. The symptom database 66 may also store values in the logs corresponding to the respective symptom that indicate whether specific manifestations of the respective symptom correspond to a respective disease diagnosis. In some cases, processing circuitry 14 may remove one or more sets of data from the symptom or disease database.

Each set of data stored by the symptom database may include one or more portions of signals measured by IMD 10, other implantable devices, other external devices, or any combination thereof. For example, IMD 10 may collect one or more of the accelerometer signal, an EGM, one or more tissue oxygenation signals (StO₂ and/or SpO₂), and one or more other signals. When IMD 10 collects a signal, IMD 10 may collect a sequence of samples corresponding to the respective signal, and the sequence of samples may represent the signal itself. Consequently, a “portion” of the signal may represent set of consecutive samples of the signal. Each set of data stored by the symptom database may include a portion of each signal of a set of signals, where each respective portion corresponds to a respective window of time. In some examples, the window of time corresponds to a time in which processing circuitry 14 has received data indicative of a user indication of a symptom. In some examples, the window of time corresponds to a time in which processing circuitry 14 detects physiological parameters that correspond to a symptom.

Processing circuitry may update the symptom database when prompted. For example, processing circuitry 14 may receive data indicative of a user indication of a symptom, collect a set of data during the time in which the symptom is being experienced, and add the set of data to the plurality of sets of data stored in the symptom database in a log corresponding to the symptom indicated by the patient.

Processing circuitry 14 may also update the symptom database on a rolling basis. For example, processing circuitry 14 may add a set of data to the plurality of sets of data stored in the symptom database when processing circuitry 14 detects physiological parameters that correspond to a symptom. Processing circuitry 14 may add the set of data reflecting the physiological parameters to the symptom database in a log corresponding to the symptom.

Processing circuitry 14 may be configured to identify symptoms based on detected physiological parameter data. Parameter values corresponding to physiological parameters may be stored in a buffer and be compared to physiological parameters stored in the symptom database. When a pattern of detected physiological parameters sufficiently matches a pattern stored in the symptom database, processing circuitry 14 may alert the patient that a symptom is being experienced and save the detected physiological parameters to the symptom database in a log corresponding to the identified symptom upon confirmation by the patient. A sufficient match may occur when the detected data matches the stored data exactly, or within a predetermined margin of error. Processing circuitry may use algorithms to determine if a match is sufficient, for example an interpolation algorithm or artificial neural network that compares detected physiological data to stored data and predicts whether the detected data is within certain bounds set by the stored data that indicate the two data sets correspond to the same symptom.

Processing circuitry 14 may set a time window based on the time or the period of time in which a symptom occurs. For example, processing circuitry 14 may set the time window to begin at a first time and end at a second time, with the first and second times being identified relative to the time or period of time in which detected physiological parameters sufficiently match physiological parameter data stored in the symptom database. In some examples, the first time may represent a time in which the detected physiological parameters first sufficiently match stored physiological parameters. In some examples, the first time may represent a time between the time in which the detected physiological parameters first sufficiently match stored physiological parameters, and the time in which the detected physiological parameters no longer sufficiently match stored physiological parameters. In some examples, the first time is a predetermined amount of time before the time or period of time in which the detected physiological parameters first sufficiently match stored physiological parameters. In some examples, the first time is a predetermined amount of time after the time or period of time in which detected physiological parameters first sufficiently match stored physiological parameters. In some examples, the second time is a predetermined amount of time after the time or period of time in which detected physiological parameters first sufficiently match stored physiological parameters, where the second time is after the first time. In some examples, the second time may represent a time at which the detected physiological parameters no longer sufficiently match stored physiological parameters, where the second time is after the first time. In any case, the time window may include at least a portion of time following the time in which the detected physiological parameters first sufficiently match stored physiological parameters.

In some cases, processing circuitry 14 may save, to the symptom database stored in a memory, a set of data including one or more signals corresponding to the time associated with sufficiently matching data sets as described above. The set of data may include a set of signal portions. Each signal portion of the set of signal portions corresponds to a respective signal collected by IMD 10 or another device and each signal portion of the set of signal portions includes data corresponding to the window of time selected by processing circuitry 14 based on the time or period of time in which the detected physiological parameter data set sufficiently matches the stored physiological parameter data set. For example, the set of data may include a portion of the accelerometer signal from the first time to the second time, a portion of the EGM collected by IMD 10 from the first time to the second time, a portion of a tissue impedance signal collected by IMD 10 from the first time to the second time, and a portion of a tissue oxygen signal collected by IMD 10 from the first time to the second time.

The symptom database may include a plurality of sets of data each corresponding to a respective symptom and the symptom database may include a plurality of “logs” configured to store one or more sets of data of the plurality of sets of data. For example, each log of the plurality of logs may be associated with a respective symptom of a plurality of symptoms. Each symptom or combination of symptoms in the database may be associated with one or more diseases. Different manifestations of a single symptom may be associated with one or more diseases, so different patterns of physiological parameter data within a symptom log, including different combinations of parameters and different changes in parameter values over time, may also be associated with different diseases. When processing circuitry 14 identifies a symptom based on a detected pattern of physiological parameter data, processing circuitry 14 may assign one or more diseases to the pattern of physiological parameter data. In some examples, the detected pattern of physiological parameter data may include: accelerometer data indicating high activity level; electrode signal data indicating a normal or slightly lower than normal heart rate. Processing circuitry 14 may compare the detected data to stored data, finding a sufficient match, and save the detected data in a log associated with a light-headedness symptom. Processing circuitry 14 may also associate the saved data to an orthostatic hypotension disease.

It may be expected that the blood pressure and/or the heart rate of patient 4 will increase in response to a body position movement such as a sit-to-stand movement. If the blood pressure and/or the heart rate of patient 4 do not increase by at least an expected amount in response to a body position movement, the patient 4 may experience light-headedness soon after completing the sit-to-stand movement. Such light-headedness may, in some examples, lead to patient 4 losing consciousness and/or falling. As such, it may be beneficial for processing circuitry 14 to analyze respective sets of data corresponding to each sit-to-stand movement detected in the accelerometer signal. That is, processing circuitry 14 may analyze the light-headedness log in the symptom database in order to determine if a sufficient match exists between detected accelerometer and other sensor data and stored accelerometer and other sensor data associated with the light-headedness symptom. When processing circuitry 14 determines that a sufficient match exists between detected sensor data and stored sensor data corresponding to light-headedness, processing circuitry 14 may determine that the patient is at risk of falling. In addition, processing circuitry 14 may notify the patient 4 through an external device 12 of the symptom and/or of the risk of falling.

Processing circuitry 14 may be configured to analyze the physiological parameter data collected by IMD 10 in order to warn patient 4 of impending symptoms. In some examples, IMD 10 may continuously collect parameter values at a predetermined frequency. IMD 10, a server, or another storage device may include a buffer or other memory structure which temporarily stores parameter values. An experienced symptom may be indicated by the patient or identified by processing circuitry 14, and a physiological parameter data set may be saved to a log in memory associated with the indicated or identified symptom. The saved data set may include a subset of data from the buffer representing physiological parameter data from a time just before the symptom was indicated or identified. At a later time, processing circuitry 14 may detect physiological parameter data that sufficiently matches the saved physiological parameter data corresponding to the time just before the symptom was experienced. In response to detecting a sufficient match, processing circuitry 14 may warn the patient 4 through an external device 12 of the impending symptom.

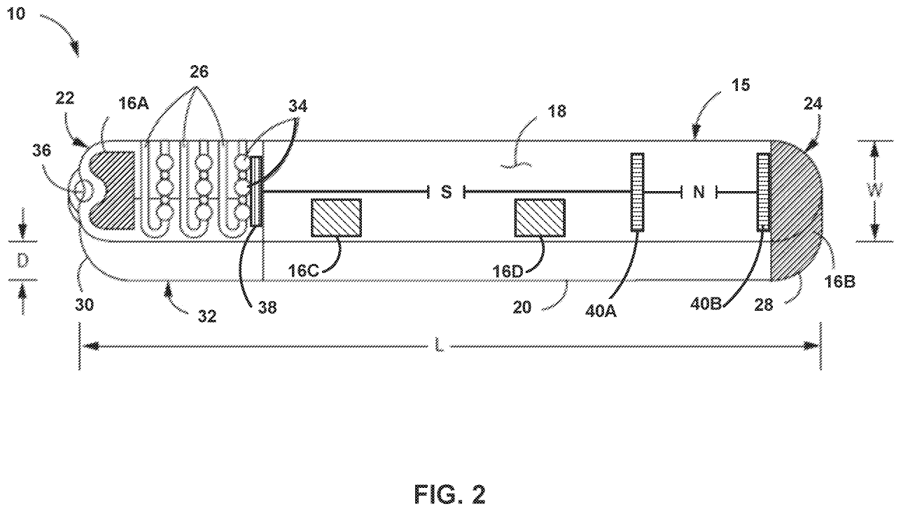

FIG. 2 is a conceptual drawing illustrating an example configuration of IMD 10 of the medical device system 2 of FIG. 1, in accordance with one or more techniques described herein. In the example shown in FIG. 2, IMD 10 may include a leadless, subcutaneously-implantable monitoring device having housing 15, proximal electrode 16A, and distal electrode 16B. Housing 15 may further include first major surface 18, second major surface 20, proximal end 22, and distal end 24. In some examples, IMD 10 may include one or more additional electrodes 16C, 16D positioned on one or both of major surfaces 18,20 of IMD 10. Housing 15 encloses electronic circuitry located inside the IMD 10, and protects the circuitry contained therein from fluids such as body fluids. In some examples, electrical feedthroughs provide electrical connection of electrodes 16A-16D, and antenna 26, to circuitry within housing 15. In some examples, electrode 16B may be formed from an uninsulated portion of conductive housing 15.

In the example shown in FIG. 2, IMD 10 is defined by a length L, a width W, and thickness or depth D. In this example, IMD 10 is in the form of an elongated rectangular prism in which length L is significantly greater than width W, and in which width W is greater than depth D. However, other configurations of IMD 10 are contemplated, such as those in which the relative proportions of length L, width W, and depth D vary from those described and shown in FIG. 2. In some examples, the geometry of the IMD 10, such as the width W being greater than the depth D, may be selected to allow IMD 10 to be inserted under the skin of the patient using a minimally invasive procedure and to remain in the desired orientation during insertion. In addition, IMD 10 may include radial asymmetries (e.g., the rectangular shape) along a longitudinal axis of IMD 10, which may help maintain the device in a desired orientation following implantation.

In some examples, a spacing between proximal electrode 16A and distal electrode 16B may range from about 30-55 mm, about 35-55 mm, or about 40-55 mm, or more generally from about 25-60 mm. Overall, IMD 10 may have a length L of about 20-30 mm, about 40-60 mm, or about 45-60 mm. In some examples, the width W of first major surface 18 may range from about 3-10 mm, and may be any single width or range of widths between about 3-10 mm. In some examples, a depth D of IMD 10 may range from about 2-9 mm. In other examples, the depth D of IMD 10 may range from about 2-5 mm, and may be any single or range of depths from about 2-9 mm. In any such examples, IMD 10 is sufficiently compact to be implanted within the subcutaneous space of patient 4 in the region of a pectoral muscle.

IMD 10, according to an example of the present disclosure, may have a geometry and size designed for ease of implant and patient comfort. Examples of IMD 10 described in this disclosure may have a volume of 3 cubic centimeters (cm³) or less, 1.5 cm³ or less, or any volume therebetween. In addition, in the example shown in FIG. 2, proximal end 22 and distal end 24 are rounded to reduce discomfort and irritation to surrounding tissue once implanted under the skin of patient 4.

In the example shown in FIG. 2, first major surface 18 of IMD 10 faces outward towards the skin, when IMD 10 is inserted within patient 4, whereas second major surface 20 faces inward toward musculature of patient 4. Thus, first and second major surfaces 18,20 may face in directions along a sagittal axis of patient 4 (see FIG. 1), and this orientation may be maintained upon implantation due to the dimensions of IMD 10.

Proximal electrode 16A and distal electrode 16B may be used to sense cardiac EGMs (e.g., cardiac ECGs) when IMD 10 is implanted subcutaneously in patient 4. In some examples, processing circuitry of IMD 10 also may determine whether cardiac EGMs of patient 4 are indicative of arrhythmia or other symptoms or diseases (e.g., heart failure, sleep apnea, or COPD). The cardiac EGMs may be stored in a memory of the IMD 10. In some examples, data derived from the EGMs may be transmitted via integrated antenna 26 to another medical device, such as external device 12. In some examples, one or both of electrodes 16A and 16B also may be used by IMD 10 to collect one or more impedance signals (e.g., a subcutaneous tissue impedance) during impedance measurements performed by IMD 10. In some examples, such impedance values detected by IMD 10 may reflect a resistance value associated with a contact between electrodes 16A, 16B, and target tissue of patient 4. Additionally, in some examples, electrodes 16A, 16B may be used by communication circuitry of IMD 10 for tissue conductance communication (TCC) communication with external device 12 or another device.

In the example shown in FIG. 2, proximal electrode 16A is in close proximity to proximal end 22, and distal electrode 16B is in close proximity to distal end 24 of IMD 10. In this example, distal electrode 16B is not limited to a flattened, outward facing surface, but may extend from first major surface 18, around rounded edges 28 or end surface 30, and onto the second major surface 20 in a three-dimensional curved configuration. As illustrated, proximal electrode 16A is located on first major surface 18 and is substantially flat and outward facing. However, in other examples not shown here, proximal electrode 16A and distal electrode 16B both may be configured like proximal electrode 16A shown in FIG. 2, or both may be configured like distal electrode 16B shown in FIG. 2. In some examples, additional electrodes 16C and 16D may be positioned on one or both of first major surface 18 and second major surface 20, such that a total of four electrodes are included on IMD 10. Any of electrodes 16A-16D may be formed of a biocompatible conductive material. For example, any of electrodes 16A-16D may be formed from any of stainless steel, titanium, platinum, iridium, or alloys thereof. In addition, electrodes of IMD 10 may be coated with a material such as titanium nitride or fractal titanium nitride, although other suitable materials and coatings for such electrodes may be used.

In the example shown in FIG. 2, proximal end 22 of IMD 10 includes header assembly 32 having one or more of proximal electrode 16A, integrated antenna 26, anti-migration projections 34, and suture hole 36. Integrated antenna 26 is located on the same major surface (e.g., first major surface 18) as proximal electrode 16A, and may be an integral part of header assembly 32. In other examples, integrated antenna 26 may be formed on the major surface opposite from proximal electrode 16A, or, in still other examples, may be incorporated within housing 15 of IMD 10. Antenna 26 may be configured to transmit or receive electromagnetic signals for communication. For example, antenna 26 may be configured to transmit to or receive signals from a programmer via inductive coupling, electromagnetic coupling, tissue conductance, Near Field Communication (NFC), Radio Frequency Identification (RFID), Bluetooth®, Wi-Fi®, or other proprietary or non-proprietary wireless telemetry communication schemes. Antenna 26 may be coupled to communication circuitry of IMD 10, which may drive antenna 26 to transmit signals to external device 12 and may transmit signals received from external device 12 to processing circuitry of IMD 10 via communication circuitry.

IMD 10 may include several features for retaining IMD 10 in position once subcutaneously implanted in patient 4. For example, as shown in FIG. 2, housing 15 may include anti-migration projections 34 positioned adjacent integrated antenna 26. Anti-migration projections 34 may include a plurality of bumps or protrusions extending away from first major surface 18 and may help prevent longitudinal movement of IMD 10 after implantation in patient 4. In other examples, anti-migration projections 34 may be located on the opposite major surface as proximal electrode 16A and/or integrated antenna 26. In addition, in the example shown in FIG. 2 header assembly 32 includes suture hole 36, which provides another means of securing IMD 10 to the patient to prevent movement following insertion. In the example shown, suture hole 36 is located adjacent to proximal electrode 16A. In some examples, header assembly 32 may include a molded header assembly made from a polymeric or plastic material, which may be integrated or separable from the main portion of IMD 10.

Electrodes 16A and 16B may be used to sense cardiac EGMs, as described above. Additional electrodes 16C and 16D may be used to sense subcutaneous tissue impedance, in addition to or instead of electrodes 16A, 16B, in some examples. In some examples, processing circuitry of IMD 10 may determine an impedance value of patient 4 based on signals received from at least two of electrodes 16A-16D. For example, processing circuitry of IMD 10 may generate one of a current or voltage signal, deliver the signal via a selected two or more of electrodes 16A-16D, and measure the resulting other of current or voltage. Processing circuitry of IMD 10 may determine an impedance value based on the delivered current or voltage and the measured voltage or current.

In the example shown in FIG. 2, IMD 10 includes light emitter(s) 38 and a proximal light detector 40A and a distal light detector 40B (collectively, “light detectors 40”) positioned on housing 15 of IMD 10. Light detector 40A may be positioned at a distance S from light emitter(s) 38, and a distal light detector 40B positioned at a distance S+N from light emitter(s) 38. In other examples, IMD 10 may include only one of light detectors 40A, 40B, or may include additional light emitters and/or additional light detectors. Collectively, light emitter(s) 38 and light detectors 40A, 40B may include an optical sensor, which may be used in the techniques described herein to determine StO₂ or SpO₂ values of patient 4. Although light emitter(s) 38 and light detectors 40A, 40B are described herein as being positioned on housing 15 of IMD 10, in other examples, one or more of light emitter(s) 38 and light detectors 40A, 40B may be positioned, on a housing of another type of IMD within patient 4, such as a transvenous, subcutaneous, or extravascular pacemaker or ICD, or connected to such a device via a lead. Light emitter(s) 38 include a light source, such as an LED, that may emit light at one or more wavelengths within the visible (VIS) and/or near-infrared (NIR) spectra. For example, light emitter(s) 38 may emit light at one or more of about 660 nanometer (nm), 720 nm, 760 nm, 800 nm, or at any other suitable wavelengths.

In some examples, techniques for determining StO₂ may include using light emitter(s) 38 to emit light at one or more VIS wavelengths (e.g., approximately 660 nm) and at one or more NIR wavelengths (e.g., approximately 850-890 nm). The combination of VIS and NIR wavelengths may help enable processing circuitry of IMD 10 to distinguish oxygenated hemoglobin from deoxygenated hemoglobin in the tissue of patient 4, since as hemoglobin becomes less oxygenated, an attenuation of VIS light increases and an attenuation of NIR decreases. By comparing the amount of VIS light detected by light detectors 40A, 40B to the amount of NIR light detected by light detectors 40A, 40B, processing circuitry of IMD 10 may determine the relative amounts of oxygenated and deoxygenated hemoglobin in the tissue of patient 4. For example, if the amount of oxygenated hemoglobin in the tissue of patient 4 decreases, the amount of VIS light detected by light detectors 40A, 40B increases and the amount of NIR light detected by light detectors 40A, 40B decreases. Similarly, if the amount of oxygenated hemoglobin in the tissue of patient 4 increases, the amount of VIS light detected by light detectors 40A, 40B decreases and the amount of NIR light detected by light detectors 40A, 40B increases.

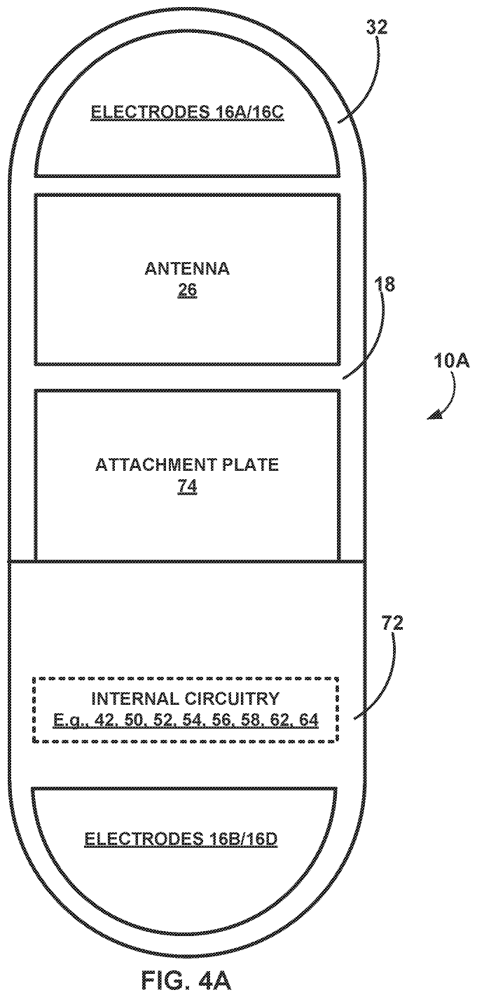

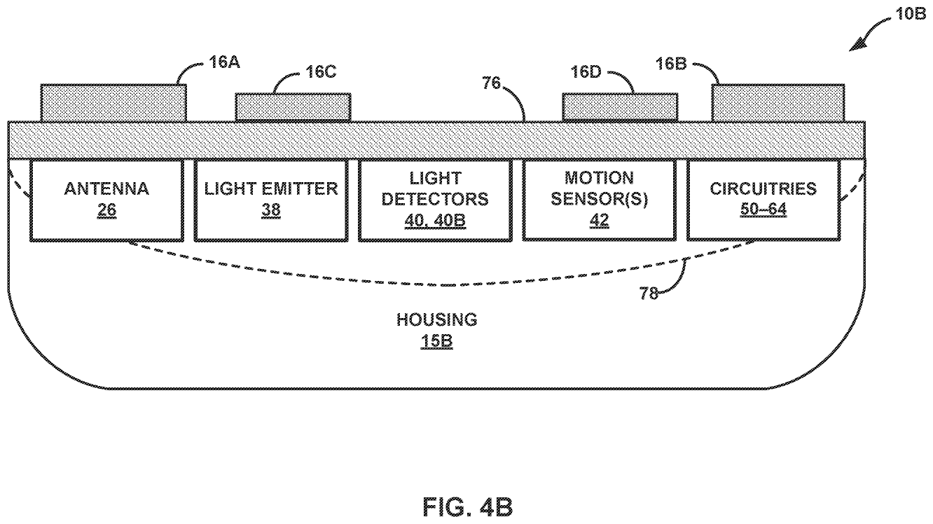

As shown in FIG. 2, light emitter(s) 38 may be positioned on header assembly 32, although, in other examples, one or both of light detectors 40A, 40B may additionally or alternatively be positioned on header assembly 32. In some examples, light emitter(s) 38 may be positioned on a medial section of IMD 10, such as part way between proximal end 22 and distal end 24. Although light emitter(s) 38 and light detectors 40A, 40B are illustrated as being positioned on first major surface 18, light emitter(s) 38 and light detectors 40A, 40B alternatively may be positioned on second major surface 20. In some examples, IMD may be implanted such that light emitter(s) 38 and light detectors 40A, 40B face inward when IMD 10 is implanted, toward the muscle of patient 4, which may help minimize interference from background light coming from outside the body of patient 4. Light detectors 40A, 40B may include a glass or sapphire window, such as described below with respect to FIG. 4B, or may be positioned beneath a portion of housing 15 of IMD 10 that is made of glass or sapphire, or otherwise transparent or translucent.

Light emitter(s) 38 may emit light into a target site of patient 4 during a technique for determining an StO₂ value of patient 4. The target site may generally include the interstitial space around IMD 10 when IMD 10 is implanted in patient 4. Light emitter(s) 38 may emit light directionally in that light emitter(s) 38 may direct the signal to a side of IMD 10, such as when light emitter(s) 38 are disposed on the side of IMD 10 that includes first major surface 18. The target site may include the subcutaneous tissue adjacent IMD 10 within patient 4.

Techniques for determining an StO₂ value may be based on the optical properties of blood-perfused tissue that change depending upon the relative amounts of oxygenated and deoxygenated hemoglobin in the microcirculation of tissue. These optical properties are due, at least in part, to the different optical absorption spectra of oxygenated and deoxygenated hemoglobin. Thus, the oxygen saturation level of the patient's tissue may affect the amount of light that is absorbed by blood within the tissue adjacent IMD 10, and the amount of light that is reflected by the tissue. Light detectors 40A, 40B each may receive light from light emitter(s) 38 that is reflected by the tissue, and generate electrical signals indicating the intensities of the light detected by light detectors 40A, 40B. Processing circuitry of IMD 10 then may evaluate the electrical signals from light detectors 40A, 40B in order to determine an StO₂ value of patient 4.

In some examples, a difference between the electrical signals generated by light detectors 40A, 40B may enhance an accuracy of the StO₂ value determined by IMD 10. For example, because tissue absorbs some of the light emitted by light emitter(s) 38, the intensity of the light reflected by tissue becomes attenuated as the distance (and amount of tissue) between light emitter(s) 38 and light detectors 40A, 40B increases. Thus, because light detector 40B is positioned further from light emitter(s) 38 (at distance S+N) than light detector 40A (at distance S), the intensity of light detected by light detector 40B should be less than the intensity of light detected by light detector 40A. Due to the close proximity of light detectors 40A, 40B to one another, the difference between the intensity of light detected by light detector 40A and the intensity of light detected by light detector 40B should be attributable only to the difference in distance from light emitter(s) 38. In some examples, processing circuitry of IMD 10 may use the difference between the electrical signals generated by light detectors 40A, 40B, in addition to the electrical signals themselves, in determining an StO₂ value of patient 4.

In some examples, IMD 10 may include one or more additional sensors, such as one or more accelerometers (not illustrated in FIG. 2). Such accelerometers may be 3D accelerometers configured to generate signals indicative of one or more types of movement of the patient, such as gross body movement (e.g., motion) of the patient, patient posture, movements associated with the beating of the heart, or coughing, rales, or other respiration abnormalities. One or more of the parameters monitored by IMD 10 (e.g., impedance, EGM) may fluctuate in response to changes in one or more such types of movement. For example, changes in parameter values sometimes may be attributable to increased patient motion (e.g., exercise or other physical motion as compared to immobility) or to changes in patient posture, and not necessarily to changes in a medical condition. Thus, in some methods of identifying or tracking a medical condition of patient 4, it may be advantageous to account for such fluctuations when determining whether a change in a parameter is indicative of a change in a medical condition.

In some examples, IMD 10 may perform an SpO₂ measurement using light emitter(s) 38 and light detectors 40. For example, IMD 10 may perform SpO₂ measurements by using light emitter(s) 38 to emit light at one or more VIS wavelengths, one more NIR wavelengths, or a combination of one or more VIS wavelengths and one more NIR wavelengths. By comparing the amount of VIS light detected by light detectors 40A, 40B to the amount of NIR light detected by light detectors 40A, 40B, processing circuitry of IMD 10 may determine the relative amounts of oxygenated and deoxygenated hemoglobin in the tissue of patient 4. For example, if the amount of oxygenated hemoglobin in the tissue of patient 4 decreases, the amount of VIS light detected by light detectors 40A, 40B increases and the amount of NIR light detected by light detectors 40A, 40B decreases. Similarly, if the amount of oxygenated hemoglobin in the tissue of patient 4 increases, the amount of VIS light detected by light detectors 40A, 40B decreases and the amount of NIR light detected by light detectors 40A, 40B increases.

Although SpO₂ measurements and StO₂ measurements may both employ the optical sensor (e.g., light emitter(s) 38 and light detectors 40) of IMD 10 to emit and sense light, SpO₂ measurements may consume significantly more energy than StO₂ measurements. In some examples, an SpO₂ measurement may consume up to 3 orders of magnitude (1,000 times) more power than an StO₂ measurement. Reasons for the energy consumption disparity include that SpO₂ measurements may require light emitter(s) 38 to be activated for up to 30 seconds, where StO₂ measurements may require light emitter(s) 38 to be activated for up to 5 seconds. Additionally, SpO₂ measurements may require a sampling rate of up to 70 Hz, whereas StO₂ measurements may require a sampling rate of up to 4 Hz.

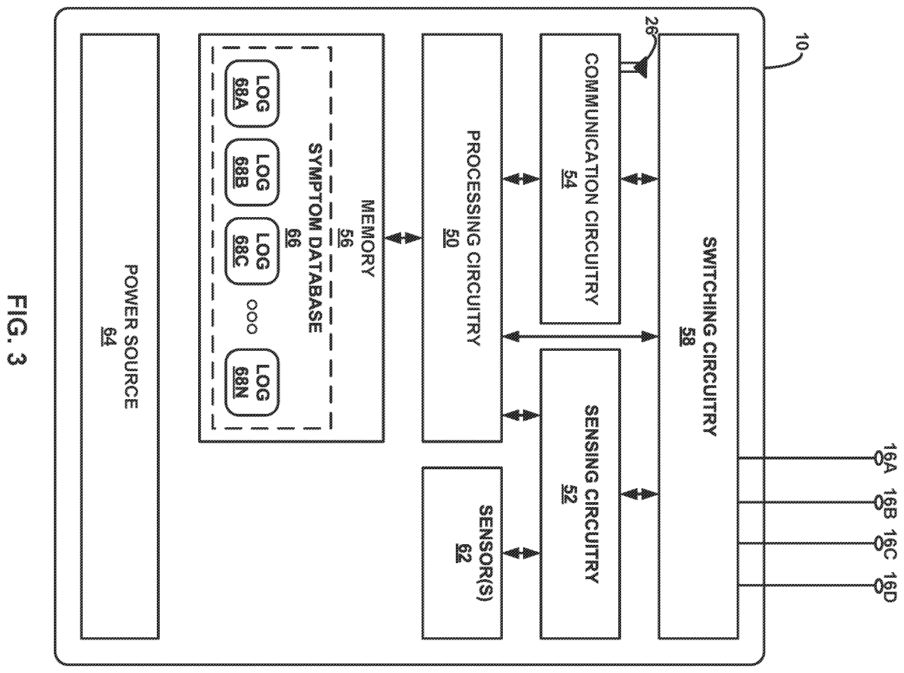

FIG. 3 is a functional block diagram illustrating an example configuration of IMD 10 of FIGS. 1 and 2, in accordance with one or more techniques described herein. As seen in FIG. 3, IMD 10 includes electrodes 16A-16D (collectively, “electrodes 16”), antenna 26, processing circuitry 50, sensing circuitry 52, communication circuitry 54, memory 56, switching circuitry 58, sensors 62, and power source 64. Memory 56 is configured to store symptom database 66 which includes logs 68A-68N (collectively, “logs 68”). Although memory 56 is illustrated as storing symptom database 66, one or more other memories may additionally or alternatively store at least a portion of symptom database 66. For example, a memory of external device 12 of FIG. 1 may be configured to store at least a portion of symptom database 66. In some examples, another memory, e.g., cloud-based, may be configured to store at least a portion of symptom database 66.

Processing circuitry 50 may include fixed function circuitry and/or programmable processing circuitry. Processing circuitry 50 may include, for example, microprocessors, DSPs, ASICs, FPGAs, equivalent discrete or integrated logic circuitry, or a combination of any of the foregoing devices or circuitry. Accordingly, processing circuitry 50 may include any suitable structure, whether in hardware, software, firmware, or any combination thereof, to perform the functions ascribed herein to IMD 10. In some examples, processing circuitry 50 may represent at least a portion of processing circuitry 14 of FIG. 1, but this is not required. In some examples, processing circuitry 50 may be separate from processing circuitry 14 of FIG. 1.

Sensing circuitry 52 and communication circuitry 54 may be selectively coupled to electrodes 16 via switching circuitry 58, which may be controlled by processing circuitry 50. Sensing circuitry 52 may monitor signals from electrodes 16 in order to monitor electrical activity of heart (e.g., to produce an EGM), and/or subcutaneous tissue impedance, the impedance being indicative of at least some aspects of patient 4's cardiac activity and/or respiratory patterns. Sensing circuitry 52 also may monitor signals from sensors 62, which may include light detectors 40, motion sensor(s) 42, and any additional sensors that may be positioned on IMD 10. In some examples, sensing circuitry 52 may include one or more filters and amplifiers for filtering and amplifying signals received from one or more of electrodes 16 and/or sensor(s) 62.

Communication circuitry 54 may include any suitable hardware, firmware, software or any combination thereof for communicating with another device, such as external device 12 or another device or sensor, such as a pressure sensing device. Under the control of processing circuitry 50, communication circuitry 54 may receive downlink telemetry from, as well as send uplink telemetry to, external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network developed by Medtronic, plc, of Dublin, Ireland.

A clinician or other user may retrieve data from IMD 10 using external device 12, or by using another local or networked computing device configured to communicate with processing circuitry 50 via communication circuitry 54. The clinician may also program parameters of IMD 10 using external device 12 or another local or networked computing device.

In some examples, memory 56 includes computer-readable instructions that, when executed by processing circuitry 50, cause IMD 10 and processing circuitry 50 to perform various functions attributed to IMD 10 and processing circuitry 50 herein. Memory 56 may include one or both of a short-term memory or a long-term memory. The memory may include, for example, RAM, DRAM, SRAM, magnetic discs, optical discs, flash memories, or forms of EPROM or EEPROM. In some examples, the memory is used to store program instructions for execution by processing circuitry 50.

Memory 56 is configured to store at least a portion of symptom database 66. Symptom database 66 includes a plurality of sets of data. Each set of data of the plurality of sets of data may, in some examples, correspond to a symptom identification detected in data collected by IMD 10. For example, at least one of the plurality of sets of data may correspond to light-headedness, atrial fibrillation, or COPD. Additionally, a plurality of sets of data may correspond to a disease identification. Any one or more of the plurality of sets of data corresponding to a disease identification may be called reference models.

In some examples, each set of data of the plurality of sets of data includes respective portions of one or more signals, where the respective portions of the one or more signals correspond to a respective time window. For example, a first set of data may include a set of signals corresponding to a first time window and a second set of data may include a set of signals corresponding to a second time window, where the first time window is different than the second time window. The first set of data may include at least one of the same signals as the second set of data. As such, the first set of data and the second set of data may include at least one overlapping signal, although the first set of data corresponds to the first time window and the second set of data corresponds to the second time window.

Symptom database 66 includes logs 68. In some examples, each of logs 68 may correspond to one or more symptoms. A set of data may be sorted into logs 68 based on one or more symptoms associated with the set of data. For example, log 68A may be associated with light-headedness, log 68B may be associated with atrial fibrillation, and log 68C may be associated with ventricular tachycardia. Logs 68C-68N may each be associated with one or more of a plurality of symptoms. When a detected set of data is associated with atrial fibrillation, memory 56 may store the detected set of data in log 68B. In some examples, processing circuitry (e.g., processing circuitry 14 of FIG. 1) may analyze one or more of logs 68 in order to determine a symptom based on detected physiological parameter values.

Power source 64 is configured to deliver operating power to the components of IMD 10. Power source 64 may include a battery and a power generation circuit to produce the operating power. In some examples, the battery is rechargeable to allow extended operation. In some examples, recharging is accomplished through proximal inductive interaction between an external charger and an inductive charging coil within external device 12. Power source 64 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

FIGS. 4A and 4B illustrate two additional example IMDs that may be substantially similar to IMD 10 of FIGS. 1-3, but which may include one or more additional features, in accordance with one or more techniques described herein. The components of FIGS. 4A and 4B may not necessarily be drawn to scale, but instead may be enlarged to show detail. FIG. 4A is a block diagram of a top view of an example configuration of an IMD 10A. FIG. 4B is a block diagram of a side view of example IMD 10B, which may include an insulative layer as described below.

FIG. 4A is a conceptual drawing illustrating another example IMD 10A that may be substantially similar to IMD 10 of FIG. 1. In addition to the components illustrated in FIGS. 1-3, the example of IMD 10 illustrated in FIG. 4A also may include a body portion 72 and an attachment plate 74. Attachment plate 74 may be configured to mechanically couple header assembly 32 to body portion 72 of IMD 10A. Body portion 72 of IMD 10A may be configured to house one or more of the internal components of IMD 10 illustrated in FIG. 3, such as one or more of processing circuitry 50, sensing circuitry 52, communication circuitry 54, memory 56, switching circuitry 58, internal components of sensors 62, and power source 64. In some examples, body portion 72 may be formed of one or more of titanium, ceramic, or any other suitable biocompatible materials.

FIG. 4B is a conceptual drawing illustrating another example IMD 10B that may include components substantially similar to IMD 10 of FIG. 1. In addition to the components illustrated in FIGS. 1-3, the example of IMD 10B illustrated in FIG. 4B also may include a wafer-scale insulative cover 76, which may help insulate electrical signals passing between electrodes 16A-16D and/or light detectors 40A, 40B on housing 15B and processing circuitry 50. In some examples, insulative cover 76 may be positioned over an open housing 15 to form the housing for the components of IMD 10B. One or more components of IMD 10B (e.g., antenna 26, light emitter 38, light detectors 40A, 40B, processing circuitry 50, sensing circuitry 52, communication circuitry 54, switching circuitry 58, and/or power source 64) may be formed on a bottom side of insulative cover 76, such as by using flip-chip technology. Insulative cover 76 may be flipped onto a housing 15B. When flipped and placed onto housing 15B, the components of IMD 10B formed on the bottom side of insulative cover 76 may be positioned in a gap 78 defined by housing 15B.

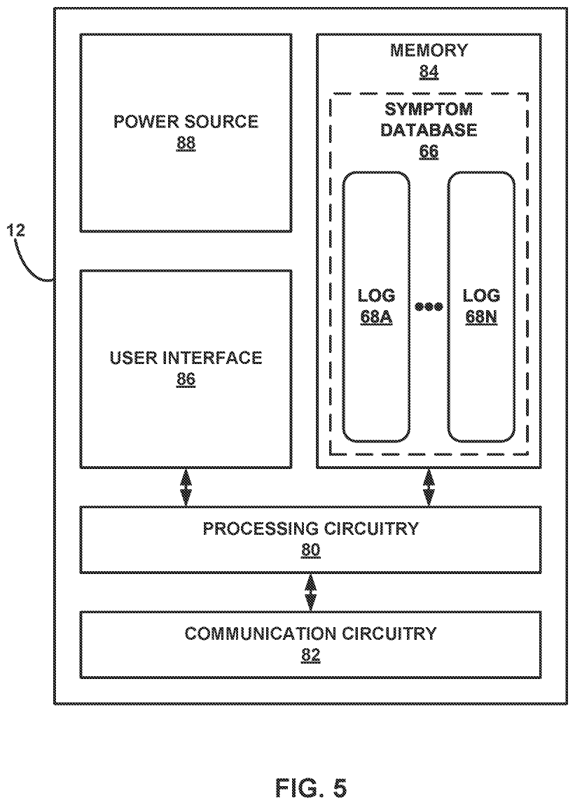

FIG. 5 is a block diagram illustrating an example configuration of components of external device 12, in accordance with one or more techniques of this disclosure. In the example of FIG. 5, external device 12 includes processing circuitry 80, communication circuitry 82, memory 84, user interface 86, and power source 88. Memory 84 is configured to store symptom database 66 which includes logs 68. Although memory 84 is illustrated as storing symptom database 66, one or more other memories may additionally or alternatively store at least a portion of symptom database 66. For example, memory 56 of IMD 10 may be configured to store at least a portion of symptom database 66. In some examples, another memory may be configured to store at least a portion of symptom database 66.

Processing circuitry 80 may include fixed function circuitry and/or programmable processing circuitry. Processing circuitry 80 may include, for example, microprocessors, DSPs, ASICs, FPGAs, equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to external device 12. In some examples, processing circuitry 80 may represent at least a portion of processing circuitry 14 of FIG. 1, but this is not required. In some examples, processing circuitry 50 may be separate from processing circuitry 14 of FIG. 1.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device.

In some examples, memory 84 includes computer-readable instructions that, when executed by processing circuitry 80, cause external device 12 and processing circuitry 80 to perform various functions attributed to IMD 10 and processing circuitry 80 herein. Memory 84 may include one or both of a short-term memory or a long-term memory. The memory may include, for example, RAM, DRAM, SRAM, magnetic discs, optical discs, flash memories, or forms of EPROM or EEPROM. In some examples, the memory is used to store program instructions for execution by processing circuitry 80. Memory 84 may be used by software or applications running on external device 12 to temporarily store information during program execution. In some examples, symptom database 66 may include one or more sets of data received from IMD 10 and sorted into logs 68.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., data corresponding to one or both of an ECG signal and an accelerometer signal) to external device 12. In turn, external device 12 may receive the collected data from IMD 10 and store the collected data in memory 84. Additionally, or alternatively, processing circuitry 80 may export instructions to IMD 10 requesting IMD 10 to update electrode combinations for stimulation or sensing.

A user, such as a clinician or patient 4, may interact with external device 12 through user interface 86. User interface 86 includes a display (not shown), such as an LCD or LED display or other type of screen, with which processing circuitry 80 may present information related to IMD 10 (e.g., EGM signals obtained from at least one electrode or at least one electrode combination, impedance signals, motion signals, an impending symptom warning, or any combination thereof). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to patient 4, receiving voice commands from patient 4, or both. Memory 84 may include instructions for operating user interface 86 and for managing power source 88.

Power source 88 is configured to deliver operating power to the components of external device 12. Power source 88 may include a battery and a power generation circuit to produce the operating power. In some examples, the battery is rechargeable to allow extended operation. Recharging may be accomplished by electrically coupling power source 88 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium ion batteries) may be used. In addition, external device 12 may be directly coupled to an alternating current outlet to operate.

FIG. 6 is a block diagram illustrating an example system that includes an access point 90, a network 92, external computing devices, such as a server 94, and one or more other computing devices 100A-100N, which may be coupled to IMD 10, external device 12, and processing circuitry 14 via network 92, in accordance with one or more techniques described herein. In this example, IMD 10 may use communication circuitry 54 to communicate with external device 12 via a first wireless connection, and to communication with an access point 90 via a second wireless connection. In the example of FIG. 6, access point 90, external device 12, server 94, and computing devices 100A-100N are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. As discussed above, IMD 10 may be configured to transmit data, such as one or more sets of data to be analyzed to external device 12. In addition, access point 90 may interrogate IMD 10, such as periodically or in response to a command from the patient or network 92, in order to retrieve parameter values determined by processing circuitry 50 of IMD 10, or other operational or patient data from IMD 10. Access point 90 may then communicate the retrieved data to server 94 via network 92.

In some cases, server 94 may be configured to provide a secure storage site for data that has been collected from IMD 10, and/or external device 12. In some cases, server 94 may assemble data in web pages or other documents for viewing by trained professionals, such as clinicians, via computing devices 100A-100N. One or more aspects of the illustrated system of FIG. 6 may be implemented with general network technology and functionality, which may be similar to that provided by the Medtronic CareLink® Network developed by Medtronic plc, of Dublin, Ireland.

Server 94 may include processing circuitry 96. Processing circuitry 96 may include fixed function circuitry and/or programmable processing circuitry. Processing circuitry 96 may include any one or more of a microprocessor, a controller, a DSP, an ASIC, an FPGA, or equivalent discrete or analog logic circuitry. In some examples, processing circuitry 96 may include multiple components, such as any combination of one or more microprocessors, one or more controllers, one or more DSPs, one or more ASICs, or one or more FPGAs, as well as other discrete or integrated logic circuitry. The functions attributed to processing circuitry 96 herein may be embodied as software, firmware, hardware or any combination thereof. In some examples, processing circuitry 96 may perform one or more techniques described herein based on one or more sets of data received from IMD 10, as examples.

Server 94 may include memory 98. Memory 98 includes computer-readable instructions that, when executed by processing circuitry 96, cause IMD 10 and processing circuitry 96 to perform various functions attributed to IMD 10 and processing circuitry 96 herein. Memory 98 may include any volatile, non-volatile, magnetic, optical, or electrical media, such as RAM, ROM, NVRAM, EEPROM, flash memory, or any other digital media. Although memory 56 is described as storing symptom database 66 and logs 68 in FIG. 3, memory 98 may additionally or alternatively store at least a portion of symptom database 66 and logs 68.

In some examples, one or more of computing devices 100A-100N (e.g., device 100A) may be a tablet or other smart device located with a clinician or physician, by which the clinician may program, receive alerts from, and/or interrogate IMD 10. For example, the clinician may access data corresponding to any one or more of an EGM, an impedance signal, a tissue perfusion signal, an accelerometer signal, and other types of signals collected by IMD 10 through device 100A, such as when patient 4 is in between clinician visits, to check on a status of a medical condition such as an experienced symptom. In some examples, the clinician may enter instructions for a medical intervention for patient 4 into an app in device 100A, such as based on the experienced symptom saved by IMD 10, external device 12, processing circuitry 14, or any combination thereof, or based on other patient data known to the clinician. Device 100A then may transmit the instructions for medical intervention to another of computing devices 100A-100N (e.g., device 100B) located with patient 4 or a caregiver of patient 4. For example, such instructions for medical intervention may include an instruction to change a drug dosage, timing, or selection, to schedule a visit with the clinician, or to seek medical attention. In further examples, device 100B may generate an alert to patient 4 based on a status of a the experienced symptom of patient 4 determined by IMD 10, external device 12, processing circuitry 14, or any combination thereof, which may enable patient 4 proactively to seek medical attention prior to receiving instructions for a medical intervention. In this manner, patient 4 may be empowered to take action, as needed, to address his or her medical status, which may help improve clinical outcomes for patient 4.

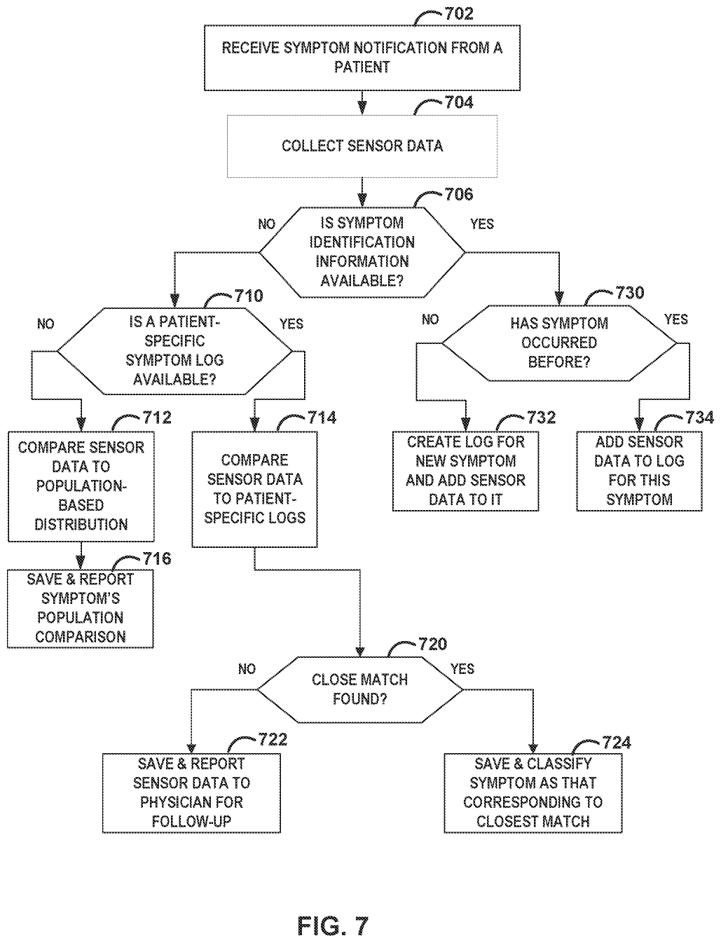

FIG. 7 is a flow diagram illustrating an example operation for generating data associated with a symptom and saving it to a database, in accordance with one or more techniques of this disclosure. FIG. 7 is described with respect to 1 MB 10, external device 12, and processing circuitry 14 of FIGS. 1-6. However, the techniques of FIG. 7 may be performed by different components of IMD 10, external device 12, and processing circuitry 14 or by additional or alternative medical device systems. Processing circuitry 14 is conceptually illustrated in FIG. 1 as separate from IMD 10 and external device 12 but may be processing circuitry of IMD 10 and/or processing circuitry of external device 12. In general, the techniques of this disclosure may be performed by processing circuitry 14 of one or more devices of a system, such as one or more devices that include sensors that provide signals, or processing circuitry of one or more devices that do not include sensors, but nevertheless analyze signals using the techniques described herein. For example, another external device (not pictured in FIG. 1) may include at least a portion of processing circuitry 14, the other external device configured for remote communication with IMD 10 and/or external device 12 via a network.

Processing circuitry 14 may receive a notification from a patient that a symptom is being experienced (702). In some examples, processing circuitry 14 may receive the notification of a symptom occurrence via communication circuitry 54 from a patient 4 interaction with external device 12. In some examples, processing circuitry 14 may receive the notification of a symptom occurrence via communication circuitry 54 from a patient 4 interaction with an external medical device, for example a fitbit or a smartwatch. The time of the symptom notification may represent a reference point for analyzing data collected by the IMD 10 and/or other devices. For example, processing circuitry 14 may determine a time before the symptom notification and a time after the symptom notification in which the identified symptom occurs.

The time after the symptom notification may be a period of time that extends until processing circuitry 14 determines that the symptom has ended. Processing circuitry 14 may compare a segment of the collected sensor data set to a baseline data set stored in memory 56. The baseline data set may represent a population-based distribution of data or patient specific data. When processing circuitry 14 determines that a segment of the collected sensor data set sufficiently matches the baseline data, it may determine that the symptom has ended, and stop saving the collected sensor data to memory 56. A sufficient match may occur when the collected data matches the baseline data exactly, or within a predetermined margin of error. Processing circuitry may use one or more comparing algorithms to determine if a match is sufficient, for example an interpolation algorithm, Siamese neural network, cross correlation, dynamic time warping, or artificial neural network. The comparing algorithm may compare collected sensor data to baseline sensor data and predict whether the detected data is within certain bounds set by the stored data that indicate the two data sets correspond to the same condition. The sensor data collected over the total time period may represent sensor data for the duration of the experienced symptom.

Processing circuitry 14 may collect sensor data from one or more sensors 62 available in a buffer memory for a time before the symptom notification, and processing circuitry 14 may generate and collect a set of sensor data from the one or more sensors 62 for a time after the symptom notification (704). The sensor data may represent one or more signals. In some examples, the one or more signals include an accelerometer signal, an impedance signal (e.g., a subcutaneous impedance signal, an intrathoracic impedance signal, and/or an intracardiac impedance signal), and the symptom represents light-headedness as indicated by the patient notification. In some examples, the one or more signals include an EGM, an impedance signal (e.g., a subcutaneous impedance signal, an intrathoracic impedance signal, and/or an intracardiac impedance signal), a tissue oxygenation signal, or any combination thereof, and the event represents a cardiac symptom identified by processing circuitry 14 in the one or more signals (i.e. ventricular tachycardia). In any case the sensor data may comprise data derived from any one or more sensors in any combination over a period of time representing the duration of the experienced symptom. The sensor data collected may be in the form of raw signal data (for example voltage frequency signals), or processing circuitry may convert the raw signal data into respective physiological parameter value data (for example a heart rate of 86 beats per minute).

The patient notification of a symptom occurrence may or may not include an identification of the symptom (706). For example, when a patient 4 indicates that patient 4 is experiencing a symptom, patient 4 may also identify it as light-headedness. In other examples, the patient 4 may indicate that patient 4 is experiencing a symptom, but not include identification information.

If symptom identification information is available, processing circuitry 14 may determine if the identified symptom has been experienced by patient 4 before (730). Processing circuitry 14 may make this determination by searching the logs 68 in the symptom database 66 in memory 56 for a log associated with the identified symptom. If a log associated with the identified system does not exist, processing circuitry 14 may determine that the identified symptom has not occurred before.

If processing circuitry 14 determines that the identified symptom has occurred before, it may save the collected sensor data from the period of time representing the duration of the experienced symptom. Such data may be saved to the log associated with the identified symptom (734).

If processing circuitry 14 determines that the identified symptom has not occurred before, it may create a log 68 in the symptom database 66 in memory 56 associated with the identified symptom (732). Processing circuitry may also save the collected sensor data to the created log associated with the identified symptom.

If symptom identification information is available, processing circuitry 14 may determine if a patient-specific symptom log is available (710). Some patients, particularly new patients, will not have symptom data in the symptom database 66 corresponding to prior experienced symptoms. However, there may be population-based distribution symptom data in the symptom database 66 in memory 56.

If a patient-specific symptom log is not available, processing circuitry 14 may compare the collected sensor data to the population-based distribution symptom data (712). While patients may experience symptoms differently from one another, there may be common elements among all experienced symptoms that are worth analyzing.

Processing circuitry 14 may save the collected sensor data to memory 56 along with comparison data showing deviations of the collected sensor data from a population-based distribution (716). Processing circuitry may also report the collected sensor data to a physician through communication circuitry 54 and external device 12. The physician may then receive data of an unidentified symptom experienced by the patient 4 and how it compares to a population-based distribution.

If a patient-specific symptom logs are available within the symptom database 66, processing circuitry 14 may compare the collected sensor data to the patient-specific symptom logs (714). Processing circuitry may then determine whether there is a sufficiently close match between the collected sensor data and sensor data saved to a log 68 associated with a symptom in the symptom database 66 in memory 56 (720). A sufficient match may occur when the collected data matches the baseline data exactly, or within a predetermined margin of error. Processing circuitry may use algorithms to determine if a match is sufficient, for example an interpolation algorithm or artificial neural network that compares collected sensor data to baseline sensor data and predicts whether the detected data is within certain bounds set by the stored data that indicate the two data sets correspond to the same condition.

If a sufficient match is not found between the collected sensor data and saved sensor data, then processing circuitry may save the collected sensor data to memory 56 and report the collected sensor data to a physician through communication circuitry 54 and external device 12 (722). The physician may then receive a notification and data of an unidentified symptom experienced by the patient 4 and be able to follow-up with the patient 4 on the experienced—yet unidentified—symptom.

If a sufficient match is found between the collected sensor data and saved sensor data, then processing circuitry may save the collected sensor data in a log 68 or update a log counter associated to the symptom with which the sufficiently matching saved sensor data is also associated (724). A log counter may maintain a count of instances of the symptom.

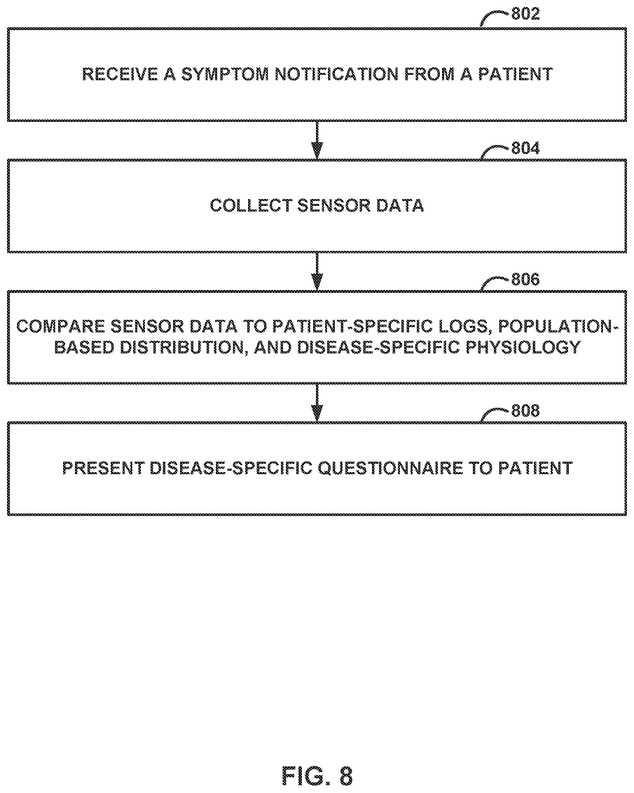

FIG. 8 is a flow diagram illustrating an example operation for generating data which may be identified as associated with more than one disease and following up with a patient for further information. A patient 4 with comorbidities may experience symptoms that are associated with more than one disease present in the patient 4. In some examples, specific manifestations of these symptoms may be uniquely associated with one of the diseases. However, in other examples the manifestations of these symptoms corresponding to one disease or another may be too similar to distinguish without further information. In any case it may be beneficial to obtain more information from a patient 4 with comorbidities when patient 4 experiences a symptom that could be associated with two or more diseases.

Processing circuitry 14 may receive a notification that a symptom is occurring from a patient 4 (802). Processing circuitry 14 may collect sensor data in accordance with one or more techniques described herein over a time period in accordance with one or more techniques described herein (804). Processing circuitry may then compare the collected sensor data to sensor data saved in memory 56 (806). The saved sensor data may comprise patient-specific symptom logs, patient-specific disease logs, population-based distributions, or any combination thereof. The saved sensor data may correspond to a disease identification and may be called a reference model. Processing circuitry may compare the collected sensor data to the saved sensor data by determining a percent difference between the collected sensor data and the saved sensor data, an absolute value difference between the collected sensor data and the saved sensor data, or determining if a sufficient match exists between the collected sensor data and the saved sensor data in accordance with one or more techniques described herein. Finally, processing circuitry 14 may present a questionnaire to the patient through communication circuitry 54 communicating with an external device 12 (808). The questionnaire may contain questions specific to the disease associated with the saved sensor data to which the collected sensor data was compared.

In one example, a patient 4 with COPD and congestive heart failure may press a patient trigger button on an external device 12 (i.e. a cell phone with an app connected to IMD 10), the sensors may indicate increased respiration rate, reduced respiration volume, no activity, increased heart rate, and no changes in fluid status. These physiological parameters may be associated with COPD or congestive heart failure. These physiological parameters may be more associated with COPD than congestive heart failure. The patient may then be sent a COPD-specific questionnaire and asked to follow-up with SpO₂ and spirometer measurements.

In another example, a diabetic patient may indicate feeling light-headed. Light-headedness may be associated with diabetes or syncope. Around the time when the symptom was reported, activity intensity was low, there were no posture changes, the heart rate did not change, and there were no potential bradycardia episodes. These physiological parameters may be more associated with blood-sugar levels from diabetes than syncope. The patient may then be presented a symptom questionnaire pertaining to diabetes to get blood-sugar information.

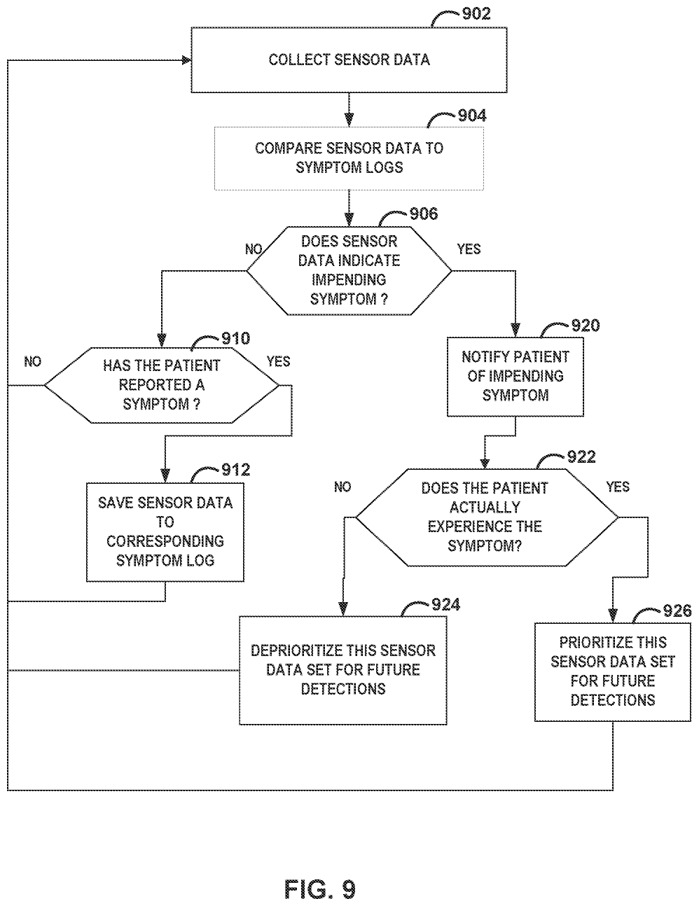

FIG. 9 is a flow diagram illustrating an example operation for predicting an impending symptom. Symptoms like dizziness and light-headedness may be followed by a danger of events like falls, and it may be beneficial to notify a patient of impending symptoms so that the patient may prepare for the danger. IMD 10 may continuously collect parameter values at a predetermined frequency. IMD 10, server 94, or another storage device may include a buffer or other memory structure which temporarily or permanently stores parameter values.

The buffer may constantly collect a certain amount of sensor data (902). Processing circuitry 14 may then compare the collected sensor data to sensor data saved in memory 56 (904). The saved sensor data may include patient-specific symptom logs. Processing circuitry may compare the collected sensor data to the saved sensor data by determining a percent difference between the collected sensor data and the saved sensor data, an absolute value difference between the collected sensor data and the saved sensor data, or determining if a sufficient match exists between the collected sensor data and the saved sensor data in accordance with one or more techniques described herein. From the comparison data, processing circuitry may use a comparing algorithm to compute a probability that a patient 4 will experience a symptom in the future. Depending on how high the computed probability of experiencing a symptom is, processing circuitry 14 may determine if the collected sensor data indicates an impending symptom (906). If the computed probability of experiencing a symptom is equal to or higher than a threshold percent (i.e. 80%), processing circuitry 14 may determine that the collected sensor data does indicate an impending symptom. If the computed probability of experiencing a symptom is lower than a threshold percent (i.e. 80%), processing circuitry 14 may determine that the collected sensor data does not indicate an impending symptom.

If the collected sensor data does not indicate an impending symptom, processing circuitry 14 may determine if data has been received indicative of a user indication of an experienced symptom (910). If the user has not indicated that a symptom is being experienced, the processing circuitry 14 continues to collect sensor data in the buffer (902). If the user has indicated that a symptom is being experienced, the processing circuitry 14 may save the collected sensor data to memory 56 (912). In some examples, the patient 4 may identify a symptom with the indication of an experienced symptom, in which case the collected sensor data may be saved to a log in the symptom database associated with the identified symptom. In other examples, the patient 4 may not have identified the symptom with the indication of an experienced symptom, in which case the collected sensor data may be saved to memory 56 and reported to a physician for follow-up.

If the collected sensor data indicates an impending symptom, processing circuitry 14 may notify a patient 4 through an external device 12 of the impending symptom (920). The patient 4 may then indicate if patient 4 actually experiences a symptom through interacting with an external device 12 (922). Processing circuitry 14 may be configured to receive the patient indication through communication circuitry 54.

If a patient 4 indicates that patient 4 did experience the impending symptom (confirmation), the saved sensor data set against which the collected sensor data set was compared may be prioritized in the comparing algorithm (926). A prioritized data set may be given more weight in the comparing algorithm, such that sufficient matches to that data set result in a higher percent chance that sufficient matches to that data set are indicative of an impending symptom.

If a patient 4 indicates that patient 4 did not experience the impending symptom (denial), the saved sensor data set against which the collected sensor data set was compared may be deprioritized in the comparing algorithm (924). A deprioritized data set may be given less weight in the comparing algorithm, such that sufficient matches to that data set result in a lower percent chance that sufficient matches to that data set are indicative of an impending symptom.

What is claimed is:

1. A medical device system comprising:
a medical device comprising one or more sensors configured to sense one or more signals that indicate one or more parameters of a patient; and
processing circuitry configured to:
receive a patient indication of an occurrence of a symptom;
determine a time period based on the patient indication;
determine a plurality of parameter values of the one or more parameters of the patient during the time period; and
save, to a database in memory, a set of data including the determined patient parameter values.

2. The medical device system of claim 1, wherein the processing circuitry is further configured to:
receive a patient identification of the symptom;
determine, based on the database in memory, if a previous occurrence of the symptom was recorded; and
responsive to determining that the symptom has occurred before, save, to the database in memory, a set of data including the determined patient parameter values in a log associated with the identified symptom; or
responsive to determining that the symptom has not occurred before, create a log associated with the identified symptom in the database in memory and save, to the database in memory, a set of data including the determined patient parameter values in the log associated with the identified symptom.

3. The medical device system of claim 1, wherein the processing circuitry is further configured to:
compare the data set to reference data sets associated with one or more symptoms from the database in memory;
determine if a sufficient match exists between the data set and at least one of the reference data sets based on the comparison; and
responsive to determining that a sufficient match exists between the data set and one of the reference data sets, save, to the database in memory, the data set or counter in a log for the symptom associated with the sufficiently matching reference data set; or
responsive to determining that a sufficient match does not exist between the data set and any of the reference data sets:
save, to the database in memory, the data set including the determined patient parameter values; and
notify a physician of the data set.

4. The medical device system of claim 1, wherein the processing circuitry is further configured to:
compare the data set to reference models associated with one or more morbidities from the database in memory;
determine if a sufficient match exists between the data set and at least one of the reference models based on the comparison; and
responsive to determining that a sufficient match exists between the data set and one of the reference models, provide follow-up questions to the patient based on the morbidity associated with the sufficiently matched reference model.

5. A medical device system comprising:
a medical device comprising one or more sensors configured to sense one or more signals that indicate one or more parameters of a patient; and
processing circuitry configured to:
determine a plurality of parameter values of the one or more parameters of the patient during a time period;
compare the determined parameter values to reference data sets associated with one or more symptoms or impending symptoms from the database in memory
determine that a sufficient match exists between the determined parameter values and one of the reference data sets;
responsive to determining that the sufficient match exists, notify the patient of the symptom associated with the one of the reference data sets; and
receive a patient confirmation or denial of the notified symptom;

6. The medical device system of claim 5, wherein the processing circuitry is further configured to:
responsive to receiving the patient confirmation of the notified symptom, prioritize the reference data set associated with the notified symptom and save, to the database in memory, the data set including the determined patient parameter values in the log associated with the notified symptom; and
responsive to receiving a denial of the notified symptom, deprioritize the reference data set associated with the notified symptom.

7. A method of collecting symptom information from a patient, the method comprising:
sensing, by a medical device comprising a set of sensors, a set of one or more signals that indicate one or more parameters of a patient;
receiving a patient indication of an occurrence of a symptom;
determining a time period based on the patient indication;
determining a plurality of parameter values of the one or more parameters of the patient during the time period; and
saving, to a database in memory, a set of data including the determined patient parameters.

8. The method of claim 7, further comprising:
receiving a patient identification of the symptom;
determining, based on the database in memory, if a previous occurrence of the symptom was recorded; and
responsive to determining that the symptom has occurred before, saving, to the database in memory, a set of data including the determined patient parameter values in a log associated with the identified symptom.

9. The method of claim 7, further comprising:
receiving a patient identification of the symptom;
determining, based on the database in memory, if a previous occurrence of the symptom was recorded; and
responsive to determining that the symptom has not occurred before, creating a log associated with the identified symptom in the database in memory and saving, to the database in memory, a set of data including the determined patient parameter values in the log associated with the identified symptom.

10. The method of claim 7, further comprising:
comparing the data set to reference data sets associated with one or more symptoms from the database in memory;
determining if a sufficient match exists between the data set and at least one of the reference data sets based on the comparison; and
responsive to determining that a sufficient match exists between the data set and one of the reference data sets, saving, to the database in memory, the data set or counter in a log for the symptom associated with the sufficiently matching reference data set.

11. The method of claim 7, further comprising:
comparing the data set to reference data sets associated with one or more symptoms from the database in memory;
determining if a sufficient match exists between the data set and at least one of the reference data sets based on the comparison; and
responsive to determining that a sufficient match does not exist between the data set and any of the reference data sets:
saving, to the database in memory, the data set including the determined patient parameter values; and
notifying a physician of the data set.

12. The method of claim 7, further comprising:
comparing the data set to reference models associated with one or more morbidities from the database in memory;
determining if a sufficient match exists between the data set and at least one of the reference models based on the comparison; and
responsive to determining that a sufficient match exists between the data set and one of the reference models, providing follow-up questions to the patient based on the morbidity associated with the sufficiently matched reference model.

13. A method of predicting symptom events in a patient, the method comprising:
sensing, by a medical device comprising a set of sensors, a set of one or more signals that indicate one or more parameters of a patient;
determining a plurality of parameter values of the one or more parameters of the patient during a time period;
comparing the determined parameter values to reference data sets associated with one or more symptoms or impending symptoms from the database in memory;
determining that a sufficient match exists between the determined parameter values and one of the reference data sets;
responsive to determining that a sufficient match exists, notifying the patient of the symptom associated with the one of the reference data sets; and
receiving a patient confirmation or denial of the notified symptom.

14. The method of claim 13, further comprising:
responsive to receiving the patient confirmation of the notified symptom, prioritizing the reference data set associated with the notified symptom and saving, to the database in memory, the data set including the determined patient parameter values in the log associated with the notified symptom.

15. The method of claim 13, further comprising:
responsive to receiving a denial of the notified symptom, deprioritizing the reference data set associated with the notified symptom.

16. A non-transitory computer-readable medium comprising instructions for causing one or more processors to, by a medical device comprising a set of sensors configured to sense one or more signals that indicate one or more parameters of a patient:
receive a patient indication of an occurrence of a symptom;
determine a time period based on the patient indication;
determine a plurality of parameter values of the one or more parameters of the patient during the time period; and
save, to a database in memory, a set of data including the determined patient parameter values.

17. The non-transitory computer-readable storage medium of claim 1 further comprising instructions for causing the one or more processors to:
receive a patient identification of the symptom;
determine, based on the database in memory, if a previous occurrence of the symptom was recorded; and
responsive to determining that a previous occurrence of the symptom was recorded, save, to the database in memory, a set of data including the determined patient parameter values in a log associated with the identified symptom; or
responsive to determining that a previous occurrence of the symptom was not recorded, create a log associated with the identified symptom in the database in memory and save, to the database in memory, a set of data including the determined patient parameter values in the log associated with the identified symptom.

18. The non-transitory computer-readable storage medium of claim 1 further comprising instructions for causing the one or more processors to:
compare the data set to reference data sets associated with one or more symptoms from the database in memory;
determine if a sufficient match exists between the data set and at least one of the reference data sets based on the comparison; and
responsive to determining that a sufficient match exists between the data set and one of the reference data sets, save, to the database in memory, the data set in a log for the symptom associated with the sufficiently matching reference data set; or
responsive to determining that a sufficient match does not exist between the data set and any of the reference data sets:
save, to the database in memory, the data set including the determined patient parameter values; and
notify a physician of the data set.

19. The non-transitory computer-readable storage medium of claim 1 further comprising instructions for causing the one or more processors to:
compare the data set to reference models associated with one or more morbidities from the database in memory;
determine if a sufficient match exists between the data set and at least one of the reference models based on the comparison; and
responsive to determining that a sufficient match exists between the data set and one of the reference models, provide follow-up questions to the patient based on the morbidity associated with the sufficiently matched reference model.

20. A non-transitory computer-readable medium comprising instructions for causing one or more processors to, by a medical device comprising a set of sensors configured to sense one or more signals that indicate one or more parameters of a patient:
determine a plurality of parameter values of the one or more parameters of the patient during a time period;
compare the determined parameter values to reference data sets associated with one or more symptoms or impending symptoms from the database in memory;
determine that a sufficient match exists between the determined parameter values and one of the reference data sets;
responsive to determining that the sufficient match exists, notify the patient of the symptom associated with the one of the reference data sets; and
receive a patient confirmation or denial of the notified symptom.

21. The non-transitory computer-readable storage medium of claim 17 further comprising instructions for causing the one or more processors to:
responsive to receiving the patient confirmation of the notified symptom, prioritize the reference data set associated with the notified symptom and save, to the database in memory, the data set including the determined patient parameter values in the log associated with the notified symptom; and
responsive to receiving a denial of the notified symptom, deprioritize the reference data set associated with the notified symptom.
